(19) United States
(12) Patent Application Publication
(10) Pub. No.: US 2006/0189985 A1
(43) Pub. Date: Aug. 24, 2006

(54) Device for providing a combination of flexibility and variable force to the spinal column for the treatment of scoliosis

(76) Inventor: David Warren Lewis, Charlottesville, VA (US)

Correspondence Address: Leo J. Aubel, 111 Rivershire Lane, Lincolnshire, IL, 60069, US

(21) Appl. No.: 11/103,687

(22) Filed: Apr. 12, 2005

Related U.S. Application Data
(60) Provisional application No. 60/651,759, filed on Feb. 9, 2005.

(51) Int. Cl. A61F 2/30

(52) U.S. Cl. 606/61

(57) ABSTRACT

Non-surgical treatments for idiopathic scoliosis include muscle stimulation therapy, chiropractic care, and the application of a variety of braces (orthotics). Surgical intervention frequently employs rigid metallic braces that prevent further flexing of the spine where applied. This invention allows flexing of the spine with long term correction of the scoliosis by application of small variable forces to supplant and counter the unbalance of the pertinent muscles. This invention may be applied to other spine problems in addition to idiopathic scoliosis as this invention permits and accommodates flexing of the spine and simultaneously supplies correcting and straightening forces.

This invention claims the priority date of provisional patent application Ser. No. 60/651759 filed Feb. 09, 2005, of the same inventor.

### FIELD OF THE INVENTION

This invention bears directly on the subject of idiopathic scoliosis offering a new approach to the treatment thereof: This invention offers an alternative to other treatments of spinal deformities and is suitable for all ages of human beings due to the inherent flexibilities that are incorporated into the concept as contrasted with the more common employ of rigid wires, braces, and fixtures.

### BACKGROUND OF THE INVENTION

The Scoliosis Research Society, dedicated to the education, research, and treatment of spinal deformity notes that idiopathic scoliosis occurs in infants, juveniles, and adolescents. The adolescent type, defined from 10-18 years of age, is the most common and represents about 80% of this type of scoliosis.

Treatment for scoliosis ranges from observation in infants to surgery in severe cases. Many infants, especially boys, grow out of the scoliosis hence close vigil should be the “treatment” initially. Juvenile idiopathic scoliosis (3-9 year olds) may rapidly progress especially in children over the age of five and may require orthotic (brace) management. Surgery is indicated if the undesirable curve of the spine is unable to be controlled by orthotic means.

Surgery may result in some foreshortening of the spine but is thought to be more desirable than allowing the curvature to increase which may cause other serious physiological problems. Frequently, surgery involves the incorporation of metallic bracing or fusion of bones that result in rigidity and therefore limits certain motions and flexing of the spine. The alternative to this rigid bracing and fusion is the subject of this invention.

This invention involves surgical intervention with the insertion of one or two different configurations of this device. One configuration of the device is attached to the pedicles of two separate vertebrae. The pedicles are singled out as having much strength but on some applications, an alternate fastening of the device will be to the transverse processes. The device, when attached to either the pedicles or the transverse processes, provides a variable force, depending upon the initial stretch or preload of the spring and of the spring rate designed into the device, and the amount of flexion resulting from rotation of the spinal column. It is this combination of flexibility and variable force that distinguishes this unique device from all other surgical implants onto the spine. This device, in one configuration, provides a tensile force, even small in value, which supplements the muscles that have been weakened or otherwise have been overcome by other unbalanced muscles acting in opposition. The long term effects of this device provide small forces that are relieved, as the undesirable spine contour is reduced, due to the diminishing of the spring force composing one main element of the invention.

A second configuration of this device may be identified principally as a compression element. This is configured so as to force apart the pedicles when attached to the two ends of the device. Note, the two pedicles selected for application of this device may be of immediately adjacent vertebrae, or not, depending upon the initial degree of curvature of the spine. With modifications to the attachment means, this same general compression configuration may be attached to transverse processes rather than pedicles. The treatment decided upon by the surgeon will determine which vertebral bodies are selected and which sections of said bodies are chosen. As before, the flexibility of this device stems from its spring rate and the amount of motion exhibited by the patient.

The two major configurations of the devices described above will be used singly or in combination depending upon the degree of curvature and location of the primary curvature of the spine. It may be necessary to employ more than one of either or both device configurations and with significantly different spring rates incorporated into the devices.

The philosophical difference in using the presently employed rigid bracing implants and the flexible devices of this invention will require planning by the surgeon. In addition, with this new invention, patients will have to be taught to restrain themselves initially as they will retain much of their initial spinal flexibility. As the forces of this device continue to interact with the forces of the patient's own muscles, the spine will slowly become more normal in contour. Simultaneously, the spring forces in this device will decrease as the muscles that have been overpowering their opposing and adjacent muscles compensate for their associated forces. It is known, physiologically, that a force applied to a muscle will ultimately yield a relaxation and an elongation of the muscle. And so the application of his invention will cause redistribution of the normal muscle activities that have been causing the spine curvature to initiate and to progress.

### BRIEF DESCRIPTION OF THE DRAWINGS

FIG. 1 is a cross section of one form of this invention generally affording a tensile force when applied to the pedicles of the spine, showing the main elements including the casing 1, the tension spring 2, the movable head 3, the base head 4, and the holes 5 through which restraining screws (not shown) will be placed, said restraining screws will transmit the forces F1 and F2.

FIG. 2 is a cross section of one form of this invention illustrating the means for applying a compressive force when affixed to the spine: showing the casing 11, the compression spring 12, the movable head 13, the base head 14, and the holes 15 through which restraining screws (not shown) will be placed, said restraining screws will transmit the forces F1 and F2.

FIG. 3 illustrates a portion of the human spine 30, a compression configuration device 31 of this invention, a second compression configuration device 32, and an extension or extraction configuration device 33 of this invention. In FIG. 3, one transverse process 34 m identified as one of many illustrated in the figure. The compression configuration and extraction configuration devices of this invention are pictured as being affixed to the respective transverse processes. However, the first choice by the surgeon for affixing the devices of this invention will be using the pedicles as they are generally stronger than the transverse processes.

FIG. 4 shows a cross section of another embodiment of this invention with the casing 50, the compression spring 51, the base head 52, the movable head 53, and a special bladder 54. One special variation or form of this invention can be recognized by the removal of the spring 51 of FIG. 4 and having the bladder 54 pressurized with air or gas. In this particular form, with the spring 51 removed, the casing 50 can be made shorter so that the device now functions as an extension rather than as a compression device. The spring rate of the device is determined by the cross sectional area of the bladder 54, the length 55, and the initial pressure in the bladder before it is moved from the position as illustrated.

FIG. 5 illustrates another extraction form of this invention employing linear springs 60 with the movable head 62 pressed nearly into or against the stop 64 of the base head 61. The linear spring may be made from rubber or one of several different forms of plastic each of which must be compatible with the human body.

FIG. 6 depicts yet another embodiment of this invention as a series of interconnected bellows that may be designed such that it acts in either the compression configuration or the extraction configuration mode or both The spring rate for this embodiment is determined by the material thickness and type, the inner and outer diameters of the individual bellow elements, and the number of bellows elements.

FIG. 7 shows the cross section of yet another embodiment of this invention with the base head 72, movable head 73, upper bladder 74, lower bladder 71, and case 70.

FIG. 8 is a cross section of another embodiment which makes use of “wave springs”. Wave springs offer certain design advantages over the more conventional helical springs including stability. This particular illustration offers one design that can function as either an extraction or compression configuration when the individual contact points of the wave s˜rings are welded together

FIG. 9 is a pictorial view that depicts the back skeletal structure of a patient afflicted with scoliosis,

FIG. 10 is also a pictorial view that depicts the inventive device super positioned on a model of the human spine to illustrate how ane inventive device would be positionable on a patient.

### DETAILED DESCRIPTION OF THE INVENTION

Certain physical deformities become apparent in the skeletal shape of the human being that can be traced to unbalances in the musculature. One classification of these defonnities is noted as idiopathic scoliosis. This invention is directed to overcoming several such physical deformities of the spine including idiopathic scoliosis. This invention introduces one or a set of forces that oppose the musculature unbalances that, with time, cause the skeletal shape to be distorted. This said distortion causes other physiological upsets to the human anatomy that may be so severe as to threaten the life of the person. If the body does not compensate for these muscle unbalances during the early growth years, certain orthotic treatments may be attempted whose purpose is to halt or stimulate other muscle counterbalances.

If normal growth does not overcome the undesirable muscle unbalances and if orthotic treatments are not successful then surgery may be necessary. In the past, the surgical approach involved either vertebral modifications including fusion or the implantation of metallic rods and braces or some combination of the two. These rods and braces, when affixed to the spine are generally rigid and therefore cause some restriction of motion of the body. Further, these implanted rods and braces are subject to revisions if they are applied to a youngster who is still growing.

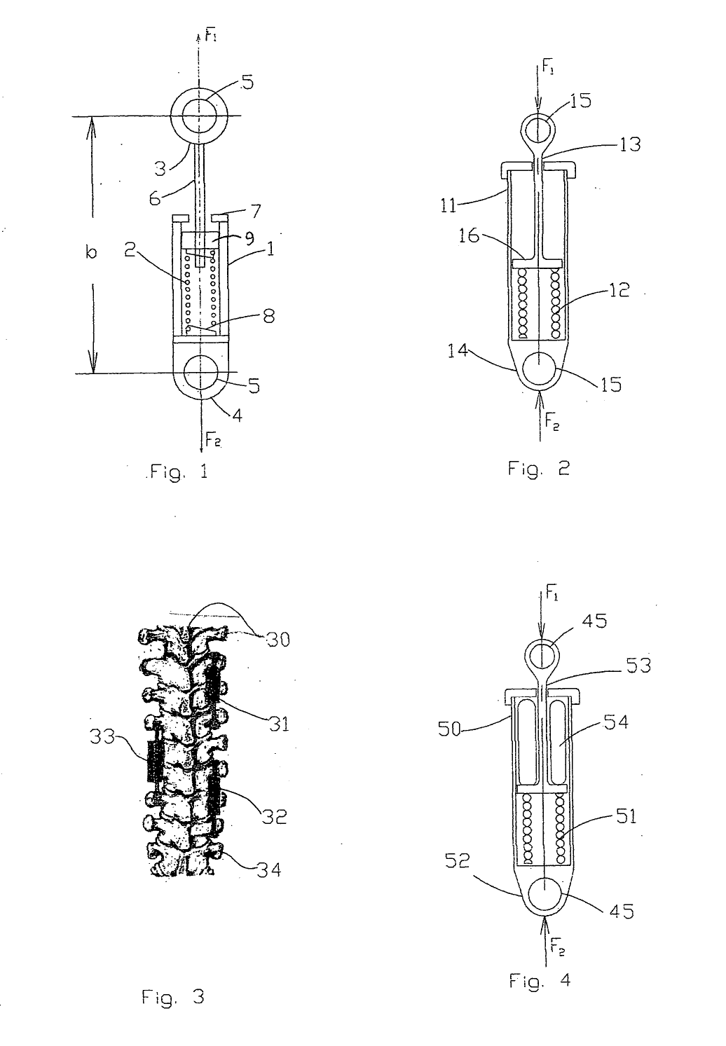

This invention provides the means for supplying variable forces that are self-adjusting as the body flexes and are directed in a manner to oppose the unbalanced musculature. The cross section of one embodiment of this invention is given in FIG. 1 whose elements can be understood to move as forces, F₁ and F₂ are applied to the opposite ends of the device through elements, typically screws (not shown), inserted in the holes 5 of the movable head 3 and the base head 4. As shown, the forces F₁ and F₂, being equal in value and oppositely directed have caused the spring 2 inside the case 1 to extend and thereby cause stresses in the spring that balance said forces F₁ and F₂. The value of the force F₁ or F₂ can be calculated as it is related to the other defining properties of the spring. For a spring constructed from wire with a circular cross section, the fundamental equation given in the book “Design Of Machine Elements” by M. F. Spotts© 1978 by Prentice-Hall, Inc. is

Sₛ=Kₛ×2×F×c³/(π×R²) in which Sₛ=shearing stress in the material in the units of pounds per square inch (or psi), Kₛ=stress multiplication factor, F=force in pounds, c=2×R/d, the spring index, d=wire diameter in inches, π=pi or approximately 3.14159, and R=mean radius of helix in inches. The spring rate k=d⁴×G/(64×R³×N), in which k is given as the pounds load for a unit deflection of the spring G=modulus of elasticity of the spring material in shear (in psi), and N=number of active coils of the spring.

As pictured in FIG. 1, F₁ and F₂ have caused the spring 2 to be extended almost to a limit as the movable head 3 has come close to bearing against the retainer stop 7. This embodiment may be designed to yield a variable force up to some specified value such as one pound or two pounds. As the length noted by “b” between the centers of the holes 5 decreases, the value of the forces F₁ and F₂ will decrease. It is this variation in the value of the forces F₁ and F₂, as the spine flexes and the distance between pedicles changes, that makes this invention unique from the alternate uses of rigid braces and the fusion process. Further, this variation in the value of the force in this device accommodates to the variation of the forces in the muscles acting on the spine.

In FIG. 1, the movable head 3 has a threaded stock 6 that may be rotated relative to the element 9 to which is affixed the spring 2. This feature affords an adjustment to the overall length of the embodiment for precise control in affixing the device to the pedicles during the surgical procedure.

FIG. 2 is a cross section of another embodiment of this invention configured so as to produce an extraction or extensive force when applied to the spine through attachment to the pedicles or transverse processes. The casing 11 incorporates the base head 14 with a hole 15, which affords the means for applying the force F₂ through an element, typically a screw (not shown), inserted in the hole 15. In this same FIG. 2, an equal force F₁ directed opposite the force F₂ just cited, is shown acting through a hole 15 of the extension head 13 and an integral piston 16, which acts on the spring 12. In this illustration, the spring 12 is compressed to its limit meaning that each helical coil is pressed against its adjacent coil. As can be understood by anyone versed in the field of solid mechanics, the forces F₁ and F₂ pictured in FIG. 2 will be acting in an opposite direction, via screws (not pictured but acting through the holes 15), onto the spine to which the screws are attached. The resulting action of the pictured form of this invention is to extract or cause additional separation of the bone elements of the spine to which the device is attached.

General Overview

As an example in selecting parameters associated with the simple spring design of FIG. 1, assume a #21 wire with d=0.0317 inch. Assume further a helix count N=10, the nominal coil diameter D=0.25 inch so that from initial touching of the coils to the overall extension of 0.633 inch, the maximum force can be calculated as F=2 pounds, and the maximum shear stress can be calculated as 43,226 psi. The maximum elongation of the spring yielding the 2.0 pound load will be 0.216 inch from which may be calculated the spring constant k=2/0.216 or k=9.26 pounds per inch. This is just an example illustrating one set of arbitrarily selected parameters for the embodiment of this invention.

With a 2.0 pound force, produced as noted by the parameters selected above, acting on a set of muscles, the muscles will stretch and thereby allow the spring to contract in overall length and the associated force acting through this invention to become smaller. Note that as muscles flex, this invention will accommodate the flexing motion by automatically changing the force produced by this device. And as the spine continues to return to the more proper natural curvature, the force(es) of the devices of this invention, assuming several are used, will be reduced.

To amplify the significance of the changing forces that this invention affords the surgeon, imagine that the portion of the spine illustrated in FIG. 3 has a curvature that may be thought of as a backward “C” or [symbol] With the extraction configuration 33 of this device pictured on the left side of the spine in FIG. 3, the tendency will be to “open” the backward “C”. Simultaneously, the compression configuration devices 31 and 32 of this device pictured on the right side of the spine in FIG. 3 will be acting in a manner to open the backward “C”. The proper selection and number of extraction and compression devices will be determined by the surgeon depending upon the degree of curvature that needs to be corrected. As each of the devices of this invention are springs yielding variable forces, as the spine in FIG. 3 becomes more straight, the values of the forces in the devices will decrease. And as can be understood by this self-accommodating combination of forces, the spine may be flexed. and when returned to the more normal attitude, the forces in the devices will return to their more normal force values. Contrast this action with the use of rigid braces, which will not allow flexing nor will they tend to correct the spine curvature over time.

As illustrated above, the sizes of the forces, being as they act over long periods of time, need not be large. A one or two pound force will have a large influence and this implies that the springs may be made from materials other than stainless steel. Certain plastics, which are materially compatible with the human body, when formed as a spring can yield a one or two pound force.

For anyone versed in the art of mechanics, FIG. 4 can be visualized as having no spring in the figure but instead having a flexible bladder filled with air or compressed gas. As the movable head 53 is extended outward from the casing 50, in the direction opposite to the direction of the force F₂, the pressure of the air or gas in the flexible bladder will increase thereby causing the configuration to act as a compression configuration device. Alternatively, if a flexible bladder replaces the spring 51 of FIG. 4, the unit will act as an extraction configuration device. Further, the use of a flexible bladder, with or without mechanical springs, will contribute damping in the operation of the devices. Said damping may be desirable especially for very active people.

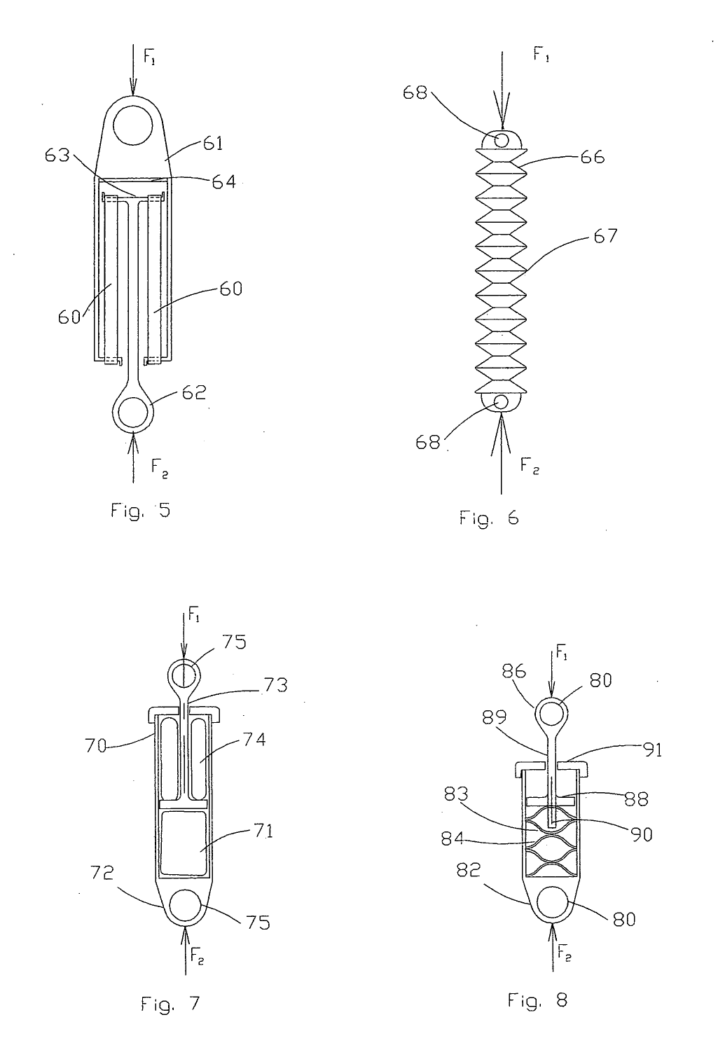

Another embodiment of this invention is given in FIG. 5 shown with the flexible elements 60 stretched by forces F₁ and F₂ acting on the movable head 62 and the base head 61. This embodiment offers the advantage of the spring rate being easily modified by changing the size of the flexing elements 60. As pictured, the flexing elements 60 have been stretched almost to the limits of the design as the extreme end 63 of the movable head has almost reached the cavity end 64 of the fixed head 61. By the selection of the material of the flexing elements 60, inherent damping can be determined for the device. As noted before, damping may be very desirable when this invention is applied to certain active human beings. A side view of this embodiment would show restraining guides, not pictured, to maintain planar alignment of the elements 61 and 62. Anyone versed in the art of design can easily visualize how this design can be “inverted” in operation to yield a compression configuration device.

An additional embodiment of this invention illustrated in FIG. 6 is through a series of interconnected bellows. Anyone versed in the art of mechanical design can understand how it may be created such that it acts in either the compression configuration or the extraction configuration mode or both. The overall spring rate for this embodiment is determined by the material thickness and type of the bellows, the inner and outer diameters of the individual bellows elements, and the number of bellows elements.

The attachment means to the spinal column pedicles will be by screws (not shown) through the holes 68, of FIG. 6. FIG. 6 illustrates the external forces F₁ and F₂ acting on the device to make it shorter thereby, when attached to the pedicles, will be acting in an extraction configuration. As described above, the means for attachment to the pedicles will be through screws but attachment to transverse processes may be accomplished by wires and with the ends of the device changed so as to provide more surface contact area.

An additional feature is shown in the embodiment of this invention in FIG. 7. With the two separate flexible bladders, that may be employed with mechanical springs (not shown), the bladders exclude flow of body fluids into and out of the main cylinder 70 of this invention as the total volume of the expansion of one bladder is compensated by the contraction of the other bladder. The pressures initially applied to the bladders, 71 and 74, will control either the extension or contraction configuration of the device. The only change in the displaced body fluid arises from the displacement of the shaft 73 as it moves in and out of the cylinder 70.

The use of a single flexible bladder with a mechanical spring will minimize the flow of body fluids. However, as noted in the calculation given above, the total size of this invention is relatively small and the total flexing, as given by the typical calculation above, is also small so that double bladders, as illustrated in FIG. 7, may not be imperative for many applications. As with the other embodiments, the attachment means to the spinal column pedicles will be by screws (not shown) through the holes 75, of FIG. 7.

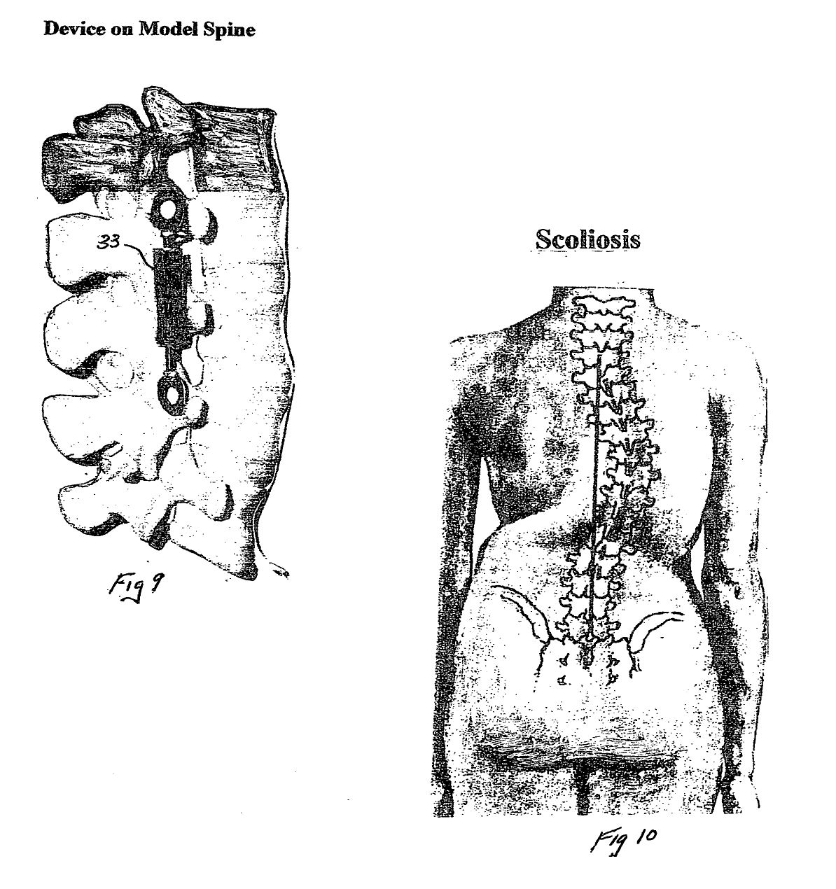

FIG. 8 is a cross section of another embodiment of this invention, which makes use of “wave springs”. Wave springs offer certain design advantages over the more conventional helical springs including stability and relative size for the equivalent displacement and force of the more common helical springs. This particular illusion offers one design that can function as either an extraction or compression configuration With one wave spring attached to the movable cap 88 and another wave spring attached to the fixed head 82, the device acts in the contraction configuration with the forces F₁ and F₂ directed as shown. By reversing the direction of the forces F₁ and F₂, this same device will act in the compression configuration. Because of this ability to act in both configurations, this embodiment is very optimal. As described and pictured in FIG. 8, one needs to visualize that the individual wave springs, such as 83 and 84, are welded at each of the respective points of contact of one spring relative to the adjacent one. This is necessary so that they may function whether being compressed or being stretched, one with respect to the other. This concept is new and is not accounted for by the manufactures of wave springs. Manufacturers wish to have slipping at the contact points of one wave spring with respect to the other. By welding these contact points, one to the other, the overall stiffness of the combination becomes greater.

As described before, the movable element 89 is threaded and matches the threaded movable cap 88. Further, the end of the threaded movable element 88 is “upset” in such a manner that will prevent the movable element 89 from being unscrewed completely from the movable element 88. This will prevent the surgeon from “accidentally” opening the unit too far and disconnecting the movable elements from the head 82. As noted before, the wave springs will not have to be large as the force levels required will be small. This will also afford the designer to employ plastic springs instead of wave springs as the total force levels will be one or two pounds.

As with other embodiments, the ring 88 is threaded such that the movable head 89 may be adjusted in length by rotating the head with respect to the ring 88. This adjustable length of the overall configuration will afford the surgeon means for proper alignment of the configuration to the vertebral bodies. Further, this adjustment means will afford the surgeon the control of the preload for either the compression or the extraction configuration. This preload adjustment means affords the surgeon an opportunity to visually change the effective curvature of the spine by the combination of more than one configuration being changed length-wise and through the adjustment of the preloads for each configuration.

The spring rate for the device of FIG. 8 is established by the number of waves, the sizes (inside and outside diameters) of the waves, the thickness of the waves, and the modulus of elasticity of the material of the waves. As can be understood by anyone versed in the art of mechanics, the combination of the properties just noted plus the adjustability by the rotation of the movable head 89 relative to the ring 88 permits a wide variety of spring rates and physical lengths of this device. And as described previously, this device may be used by attachment to pedicles and/or transverse processes of the vertebral bodies.

As many possible embodiments may be made of the invention without departing from the scope thereof, it is to be understood that all matter herein set forth and shown in the accompanying drawings is to be interpreted as illustrative and not in a limiting illuminating sense.

1-9. (canceled)

10. A device for affixing to the vertebra of a spinal column of a human body for treatment of scoliosis and other spinal deformities which vertebra includes a plurality of vertebral bodies having pedicles, said device comprising,
a) a series of flexible wave springs;
b) means for attachment of said springs to selected pedicles of vertebral bodies to provide forces that extend length wise of the spine;
c) said springs being designed to provide an initial force to said vertebral bodies through said means for attachment; and,
d) said springs exerting variable flexible forces as the vertebral bodies move in response to the flexing of the spinal column.

11. A device affixing to a vertebra of a spinal column of the human body for treatment of scoliosis which vertebra includes a plurality of vertebral bodies having pedicles, said device comprising,
a) a plurality of flexible bladders;
b) means including screws for attachment of said bladders to selected pedicles of the vertebral bodies;
c) said bladders providing variable forces through said attachment means to said vertebral bodies as said vertebral bodies move through general flexing of the spinal column; and
d) said bladders having prescribed internal pressures to provide variable forces initially established by the device design.

12. A device affixing to a vertebra of a spinal column of the human body for treatment of scoliosis and other spinal deformities which vertebra includes a plurality of vertebral bodies having pedicles, said device comprising,
a) a series of wave springs;
b) means including screws or wires for attachment of said springs to the transverse processes of vertebral bodies;
c) said springs exerting variable forces through said means for attachment to said vertebral bodies, said variable forces being dependent on the general flexing of the spinal column; and
d) said springs being designed to provide an initial force and an initial pre-load to establish the effective variable forces wherein said forces may selectively act in an extraction mode or a compression mode.

13. A device as in claim 12 further comprising
a) means for attaching individual wave springs to one another at selected points to enable forces to be transmitted through said series of wave springs in either a compression mode or a tension mode.

14. A device affixing to a vertebra of a spinal column of the human body for treatment of scoliosis and other spinal deformities which vertebra includes a plurality of vertebral bodies having pedicles, said device comprising,
a) a series of helical springs;
b) means including screws or wires for attachment of said springs to the transverse processes of vertebral bodies;
c) said springs exerting variable forces through said means for attachment to said vertebral bodies dependent on the general flexing of the spinal column;
d) said springs selectably acting in compression and extraction force modes;
e) said springs providing a predetermined initial force and an initial pre-load to establish the effective variable forces; and
f) said springs being compositions of metal, rubber and or plastics compatible with the chemistry of the human body.

15. A device as in claim 14 wherein the forces are in the two to ten pound range effective for spinal application.
